(19) United States
(12) Patent Application Publication
(10) Pub. No.: US 2002/0002170 A1
(43) Pub. Date: Jan. 3, 2002

(54) Piperidine and piperazine derivatives as inhibitors of the abeta fibril formation

(76) Inventors: Rainer M. Luond, Therwil (CH); Markus Banziger, Bubendorf (CH); Peter Frey, Bern (CH)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/906,249

(22) Filed: Jul. 16, 2001

Related U.S. Application Data
(63) Continuation of application No. PCT/EP00/01000, filed on Feb. 8, 2000.

(30) Foreign Application Priority Data
Feb. 10, 1999 (GB) 9902938.1

(51) Int. Cl. A61K 31/496; A61K 31/48; A61K 31/473; C07D 457/00

(52) U.S. Cl. 514/253.02; 514/253.03; 514/288; 514/290; 546/101; 546/67; 544/361

(57) ABSTRACT

The invention provides a compound of formula I

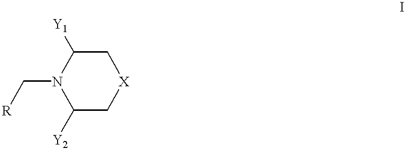

wherein R, X, Y₁ and Y₂ are as defined in the description, and a process for preparing them. The compounds of formula I are useful as pharmaceuticals.

The present invention relates to novel piperidine and piperazine derivatives, their preparation, their use as pharmaceuticals and pharmaceutical compositions containing them.

More particularly the invention provides a compound of formula I

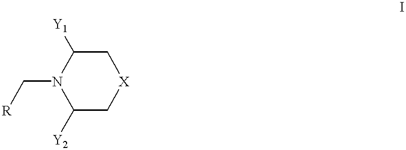

wherein

X is

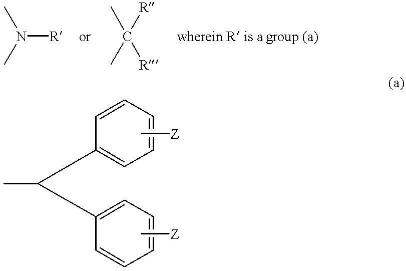

and either R″ is H or OH and R′″ is a group (b), (c) or (d)

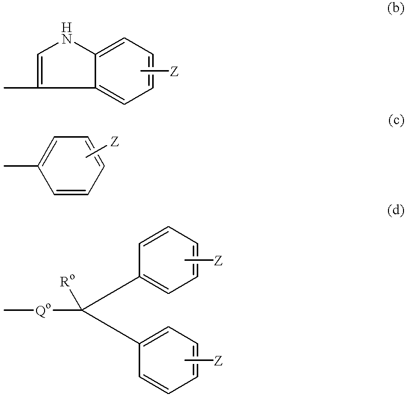

or R″ and R′″ each are a group (c),

wherein Z is H, halogen, trifluoromethyl, (C₁₋₄)alkyl or (C₁₋₄)alkoxy, Q° is —O—, —NH—CO— or a single bond and R° is hydrogen or hydroxy,

Y₁ and Y₂ are H or, when X is

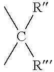

wherein R″ is H and R′″ is a group (d), Y₁ and Y₂ can also form together a —CH₂—CH₂— bridge, and

R is a group (e) or (f)

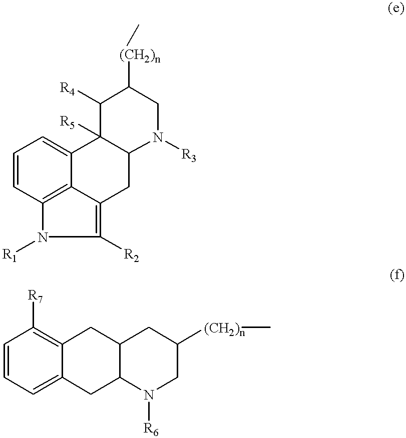

wherein

n is 0 to 3

R₁ is H, (C₁₋₄)alkyl or —SO₂—CH₃

R₂ is H, halogen, (C₁₋₄)alkyl, (C₁₋₄)alkoxy, (C₁₋₄)alkylthio or phenyl,

R₃ is H, (C₁₋₄)alkyl or a group (g)

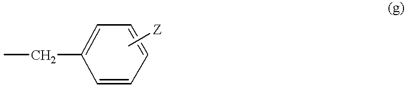

wherein Z is as defined above,

R₄ and R₅ each are H or together form a bond, or R₄ is H and R₅ is (C₁₋₄)alkoxy,

R₆ is (C₁₋₄) alkyl or a group (g) and

R₇ is (C₁₋₄) alkoxy,

in free base or acid addition salt form.

Halogen is fluorine, chlorine, bromine or iodine, preferably bromine, fluorine or chlorine.

Any alkyl, alkoxy and alkylthio radicals preferably are straight chain radicals. They preferably have 1 to 3 carbon atoms, more preferably they are methyl, methoxy and methylthio groups.

On account of the asymmetrical carbon atoms which may be present in the compounds of formula I and their salts, the compounds may exist in optically active form or in form of mixtures of optical isomers, eg in form of racemic mixtures. All optical isomers and their mixtures including the racemic mixtures are part of the present invention.

In a further aspect, the invention provides a process for the production of the compounds of formula I and their salts, whereby

a) a compound of formula II

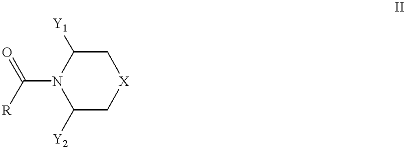

wherein X, Y₁, Y₂ and R are as defined above, is reduced or

b) a compound of formula III

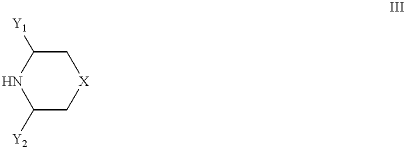

wherein X, Y₁ and Y₂ are as defined above, is reacted with a compound of formula IV

R—CH₂—Q IV

wherein R is as defined above and Q is a halogen, mesyl or tosyl,

and the compounds of formula I thus obtained are recovered in free base or acid addition salt form.

Processes (a) and (b) are conventional reduction and N-substitution reactions which can be effected according to well-known methods, eg as described in the examples.

The intermediates of formula II can be obtained from compounds of formula III by conventional amide formation, eg with acids of formula R—COOH, R being as defined above, or reactive derivatives thereof, for example sodium salts.

According to a preferred embodiment, the sodium salt is prepared from the corresponding methyl ester in free base or acid addition salt form, e.g. as p-toluene sulfonic acid salt, obtained as described in example 6.

More generally the process described in example 6 is particularly advantageous for the preparation of methyl esters of formula V in free base or acid addition salt form

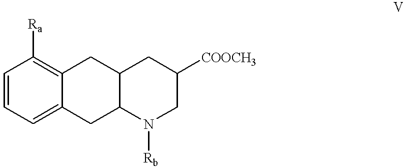

wherein Rₐ is hydroxy or (C₁₋₄)alkoxy and R_{b} is optionally substituted (C₁₋₄)alkyl, for example methyl, isopropyl or a group (g) as defined above, starting from a compound of formula VI

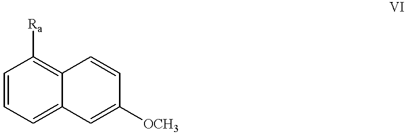

wherein Rₐ is as defined above, and ethoxymethylene cyano-acetic acid.

Methylesters of formula V are valuable intermediates for the preparation of pharmaceutically active agents which, in addition to compounds of formula I wherein R is a group (f), include for example quinagolide (Norprolac®) and [3R,4aR,10aR]-1,2,3,4,4a,5,10,10a-octahydro-6-methoxy-1-methyl-benz[g]quinoline-3-carboxylic-acid 4(4-nitro-phenyl)-piperazine-amide.

The starting materials of formula III, IV and VI are known or may be produced in analogous manner to known procedures, e.g. as described in the examples.

Compounds of formula I in optically pure form can be obtained from the corresponding racemates according to well-known procedures. Alternatively, optically pure starting materials can be used.

Acid addition salts may be produced in known manner from the free base forms and vice-versa. Suitable pharmaceutically acceptable acid addition salts for use in accordance with the present invention include for example the fumarate, the naphthalene-1,5-disulfonate, the succinate and the m-tartrate.

The compounds of formula I and their pharmaceutically acceptable acid addition salts hereinafter referred to collectively as “agents of the invention”, exhibit pharmacological activity and are, therefore, useful as pharmaceuticals.

In particular the agents of the invention inhibit the formation of β-amyloid (Aβ) peptide into neurotoxic fibrils, thereby acting to prevent or slow down the accumulation of amyloid protein deposits in the brain.

The activity of the agents of the invention in inhibiting Aβ fibril formation is determined in vitro in the following assays:

a) Thioflavin T fluorescence assay

Fibril formation at 37° C. in the presence or the absence of the inhibitors is measured by the increase in thioflavine T fluorescence (Levine et al., 1993, 1997). All experiments are carried out with Aβ 1-40, obtainable for example from BACHEM. To 100 μM Aβ in a buffer containing 25 mM phosphate and 120 mM NaCl plus 3 μM thioflavine T, final pH 7.4, equimolar and subequimolar amounts of inhibitor are added (ratio inhibitor: Aβ 1:1, 1:3, 1:10). The assay is carried out at 37° C. in 96-well fluorescence plates. Fluorescence measurements (excitation wavelength 450 nm, emission wavelength 482 nm) are done at daily intervals for at least 10 days. A thioflavine T fluorescence signal can only be observed in the presence of fibrillar Aβ. The time-point of the fibril formation is therefore assessed indirectly, by taking the time of the first statistically significant increase of the fluorescence signal over background (tc, time-point for the control). The activity of a test substance in delaying the fibril formation can be measured, for example, by dividing the time t of the first statistically significant increase in the fluorescence signal over background in the presence of the inhibitor, by the time tc of the control without inhibitor (t/tc).

In the seeded fluorescence assays, 1% of Aβ fibrils stemming from previous experiments are added to the incubation solutions. Seeding accelerates fibril formation considerably. Substances active in this test are thought to block the addition of Aβ monomers/oligomers to fibrils.

With the agents of the invention, amyloid fibril formation is significantly delayed in these assays.

b) Turbidity assay

Aβ fibril formation in vitro is greatly accelerated by shaking the solution. The progressive fibril formation can be assessed by turbidity measurements at OD 405 nm. All experiments are carried out with Aβ 140, obtainable for example from BACHEM. To 100 μM Aβ in a buffer containing 20 mM phosphate and 120 mM NaCl, final pH 7.4, equimolar and subequimolar amounts of inhibitor are added (ratio inhibitor: Aβ 1:1, 1:3, 1:10). The assay is carried out in 96-well plates shaken at room temperature. Turbidity measurements are done in 10-minute intervals for 2.5 hours, then in 30-minute intervals for another 1.5 hours. Turbidity is assessed by measuring the optical density (OD) at 405 nm. The time-point of the fibril formation is assessed by taking the time of the first statistically significant increase of the OD₄₀₅ₙₘ signal over background (tc, time-point for the control). The activity of a test substance in delaying fibril formation can be measured, for example, by dividing the time t of the first statistically significant increase in the OD₄₀₅ₙₘ signal over background in the presence of the inhibitor, by the time tc of the control without inhibitor (t/tc). In the seeded turbidity assays, 1% of Aβ fibrils stemming from previous turbidity experiments are added to the incubation solutions. Seeding furthermore accelerates the appearance of turbidity by a factor of 1.5 to 2. Substances active in this test are thought to block the addition of Aβ monomers/oligomers to fibrils.

With the agents of the invention, amyloid fibril formation is significantly delayed in these assays.

The agents of the invention are therefore useful for the treatment of any condition responsive to Aβ accumulation or deposition in brain tissue in patients suffering from or susceptible to said conditions. More particularly the agents of the invention are useful for the treatment of amyloidoses, such as Alzheimer's disease, Down's syndrome and multi-infarct dementia, or cerebral haemorrhage with amyloidosis.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.01 to about 100, preferably from about 0.1 to about 50 mg/kg animal body weight. In larger mammals, for example humans, an indicated daily dosage is in the range from 1 to about 500, preferably from about 5 to about 300 mg of an agent of the invention conveniently administered, for example, in divided doses up to four times a day or in sustained release form.

The agent of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a pharmaceutical, e.g. for the treatment of conditions resulting from Aβ accumulation or deposition in brain tissue.

The present invention furthermore provides a pharmaceutical composition comprising an agent of the invention in association with at least one pharmaceutical carrier or diluent. Such compositions may be manufactured in conventional manner. Unit dosage forms contain, for example, from about 0.25 to about 150, preferably from about 1 to about 25 mg of a compound according to the invention.

Moreover the present invention provides the use of an agent of the invention, for the manufacture of a medicament for the treatment of any condition mentioned above.

In still a further aspect the present invention provides a method for the treatment of any condition mentioned above, in a subject in need of such treatment, which comprises administering to such subject a therapeutically effective amount of an agent of the invention.

The following examples illustrate the invention. The temperatures are given in degrees Celsius and are uncorrected.

### EXAMPLE 1

### [3S,4aR,10aR]-3-{2-[4,4-Bis-(4-methoxy-phenyl)-piperidin-1-yl]-ethyl}-6-methoxy-1-methyl-1,2,3,4,4a,5,10,10a-octahydro-benzo[g]quinoline

a) A mixture of [3R,4aR,10aR]-methanesulfonic acid 6-methoxy-1-methyl-1,2,3,4,4a,5,10,10a-octahydro-benzo[g]quinolin-3-ylmethyl ester (12.3 g, 36.3 mmol) and potassium cyanide (4.72 g, 72.6 mmol) in DMSO (160 ml) is heated at 100° for 1 hour in the presence of catalytic amounts of potassium iodide (50 mg). The yellow cold solution is diluted with ethyl acetate (600 ml) and washed thoroughly with water and brine. The organic phase is dried with sodium sulfate, decolorized with activated charcoal, filtered and concentrated in vacuo to give 7.9 g (29 mmol, 81%) of the nitrile as a white solid. m.p. 130-133°. TLC 0.2 (silica, 10:1 ethyl acetate:MeOH).

b) A solution of the above nitrile (7.7 g, 28.5 mmol) in dry methanol (200 ml) is saturated with dry, gaseous HCl under external cooling and is then refluxed for 3.5 hours. The cold reaction mixture is carefully neutralized with a sat. KHCO₃-solution. The milky residue is diluted with ethyl acetate (500 ml) and washed with water and brine. The organic phase is dried with sodium sulfate, filtered and concentrated in vacuo to give the methyl ester as a white solid 8.3 g (27.3 mmol, 96%). m.p. 105-108°. TLC 0.18 (silica, 10:1 ethyl acetate:MeOH).

c) The above ester (8.2 g, 26.9 mmol) is dissolved in 40 ml THF and 40 ml MeOH and is treated carefully with NaOH (1M, 37.6 ml, 37.6 mmol) at room temperature. The final product is precipitated with the addition of tert.-butyl-methylether. The chilled precipitate is filtered, washed with cold tert.-butyl-methylether/MeOH 3:1 (25 ml) and dried at 50° in the oven at reduced pressure. White powder 8.6 g (27.6 mmol, 100%). m.p. 264-268 °. FAB-MS: 334 (M+Na)⁺, 312 (M+1)⁺.

d) The so obtained [3S,4aR,10aR]-(6-methoxy-1-methyl-1,2,3,4,4a,5,10,10a-octahydro-benzo[g]quinoline-3-yl)-acetic acid sodium salt (0.5 g, 1.6 mmol) is suspended in dry DMF (50 ml) and THF (50 ml) at 0°. N-(3-Dimethylaminopropyl)-N′-ethylcarbodiimid hydrochloride (340 mg, 1.76 mmol) and hydroxybenzotriazole (240 mg, 1.76 mmol) are added and the solution is stirred for 45 min. 4,4-Bis-(4-methoxy-phenyl)-piperidine (0.476 g, 1.6 mmol) is added and the solution is kept at room temperature for 22 hours. The reaction mixture is quenched with sat. NaHCO₃, diluted with ethyl acetate and washed carefully with water and brine. The organic phase is dried over sodium sulfate, filtered and concentrated in vacuo. White foam 0.6 g (1.0 mmol, 66%). TLC 0.5 (silica, 60:5:1 dichloromethane:MeOH:AcOH), ESI-MS: 569.

e) To a solution of the above amide (0.6 g, 1.0 mmol) in THF (30 ml) is added lithium aluminum hydride (0.12 g, 3.2 mmol) at room temperature. After 1 day sat. potassium carbonate solution (2.5 ml) is added followed by 2 spatula of hyflo. The white suspension is filtered and washed with ethyl acetate. The filtrate is washed with water and brine, the organic phase is dried over sodium sulfate, filtered and concentrated in vacuo. To the yellow residue dissolved in ethanol is added succinic acid (0.33 g, 2.8 mmol, 1.5 aeq.) to form the succinate salt. The white solid is recrystallized once from ethanol. 0.66 g (78%). M.p. 138-142° (disuccinate). ESI-MS: 555 [MH]⁺. [a]_{D}−46.1 (c=0.915, H₂O).

The following compounds of formula I are prepared analogously to Example 1:

### EXAMPLE 2

### [3S,4aR,10aR]-3-{2-[4-(1-H-Indol-3-yl)-piperidin-1-yl]-ethyl}-6-methoxy-1-methyl-1,2,3,4,4a,5,10,10a-octahydro-benzo[g]quinoline

M.p. 215-217° (disuccinate). ESI-MS: 458 [MH]⁺. [α]_{D}−91.9 (c=0.785, DMF).

### EXAMPLE 3

### [3S,4aR,10aR]-4-(4-Chloro-phenyl)-1-[2-(6-methoxy-1-methyl-1,2,3,4,4a,5,10,10a-octahydro-benzo[g]quinolin-3-yl)-ethyl]-piperidin-4-ol

M.p. 212-215° (disuccinate). ESI-MS: 469 [MH]⁺. [α]_{D}−58.1 (c=0.79, DMF).

### EXAMPLE 4

### [3S,4aR,10aR]-3-[2-(endo-3-Benzhydryloxy-8-aza-bicyclo[3.2.1]oct-8-yl)-ethyl]-6-methoxy-1-methyl-1,2,3,4,4a,5,10,10a-octahydro-benzo[g]quinoline

M.p. 176-178° (disuccinate). ESI-MS: 551 [MH]⁺. [α]_{D}−51.4 (c=1.01, DMF).

### EXAMPLE 5

### [3S,4aR,10aR]-3-[2-(4-Benzhydryl-piperazin-1-yl)-ethyl]-6-methoxy-1-methyl-1,2,3,4,4a,5,10,10a-octahydro-benzo[g]quinoline

M.p. 140-148° (free base). ESI-MS: 510 [MH]⁺. [α]_{D}−78.9 (c=0.73, DMF).

### EXAMPLE 6

### [3S,4aR,10aR]-3-(endo-3-Benzhydryloxy-8-aza-bicyclo[3.2.1]oct-8-ylmethyl)-6-methoxy-1 -methyl-1,2,3,4,4a,5,10,10a-octahydro-benzo[g]quinone

a) 2-Cyano-3-(3,8-dimethoxy-naphthalene-2-yl)-acrylicacid ethylester

1,6-Dimethoxynaphthalene (60.24 g, 320 mmol) is dissolved in 464 ml THF and cooled to −20°. Then 107 g hexyllithium (33% solution in hexane, 383 mmol) are added and the mixture is stirred at 0° for 3 h. This reaction mixture is then cooled to −70° and then a solution of ethoxmethylenecyanoacetate (62.24 g, 368 mmol) in 310 ml THF is added, at such a rate that the temperature does not rise above −65°. After the addition is complete the reaction mixture is stirred for an additional hour at −65°, then warmed to −20° and finally 1M sulfuric acid (220 ml) are added. During the addition the product starts precipitating. The mixture is stirred for 0.5 h at 0°, then the product is filtered off and dried in vacuo at 60°.

This crude product is recristallized from toluene (160 ml). 54.6 g (175 mmol, 55%). m.p. 159°-161°; ¹H-NMR (CD₂Cl₂: 400 MHz): 1.4 (t, 3H), 4.03 (s, 3H, OCH₃), 4.08 (s, 3H, OCH₃), 4.41 (q, 2H), 6.78 (d, 1H, H-C7), 7.18 (s, 1H, H-C4), 7.35 (d, 1H, H-C5), 7.50 (t, 1H, H-C6), 8.81 (s, 1H, H-C3), 9.22 (s, 1H, H-C1).

b) 2-Aminomethyl-3-(3,8-dimethoxy-naphthalene-2-yl)-propionic acid

A suspension of 2-Cyano-3-(3,8-dimethoxy-naphthalene-2-yl)-acrylicacid ethylester (60 g, 193 mmol) in 900 ml ethanol is hydrogenated in the presence of 12 g Pt/C (5%) and sulfuric acid (30 g) at 50° and 10 bar. After the theoretical hydrogen consumption (ca. 4 h) the hydrogenation is stopped. The catalyst is filtered off, washed with ethanol and the filtrate is concentrated to a volume of 540 ml. Then 540 ml water are added, followed by lithium hydroxide monohydrate (34.85 g, 831 mmol). This mixture is heated to reflux for 3 h, then the pH is adjusted to pH 8-8.5 by addition of acetic acid (30.4 g). The product precipitates, the mixture is cooled to 20° and the product is filtered. The wet filtercake is suspended in water (540 ml) and ethanol (540 ml), dissolved as the lithium salt, by addition of lithiumhydroxide monohydrate (8.92 g, 213 mmol), heated to 60° and then the pH is adjusted to pH 8.0-8.5 by addition of acetic acid (12.8 g, 213 mmol). The product precipitates, the suspension is cooled to 20°, filtered, washed with ethanol/water and dried in vacuo at 80°. 46.1 g (159 mmol, 83%). ¹H-NMR (CD₃OD/NaOD: 200 Mhz): 2.58-3.20 (m, 5H), 3.92 (s, 3H, OCH₃), 3.96 (s, 3H, OCH₃), 6.65-6.77 (m, 1H, H-C6), 7.12 (s, 1H, H-C4), 7.22-7.32 (m, 2H, H-C5 und H-C7), 8.00 (s, 1H, H-C1).

c) 6-Methoxy-2,3,4,4a,5,10-hexahydro-benzo[.g.]quinoline-3-carboxylic acid hydrochloride

2-Aminomethyl-3-(3,8-dimethoxy-naphthalene-2-yl)-propionic acid (40 g, 138.2 mmol) is suspended in THF (400 ml) and t-butanol (20.48 g, 276.3 mmol). This suspension is cooled to −70° and ammonia (150 g) is condensed into the mixture, followed by the portionwise addition of lithium metal (2.3 g, 331.4 mmol). After 1.5 h the cooling bath is removed and ammonia is evaporated. To the suspension water (270 ml) is added and the THF and t-butanol are distilled off at 50° in vacuo. This aqueous solution is then poured into conc. hydrochloric acid (116 g), at a temperature below 10°. The desired product precipitates, the mixture is stirred for 4 h in the ice bath, then the product is filtered and washed with 2 M hydrochloric acid (72 ml), followed by ethyl acetate (100 ml). 39.7 g (134 mmol, 97%). ¹H-NMR (D6-DMSO: 400 Mhz): 1.60-1.75 and 1.90-2.00 and 2.15-2.25 and 2.35-2.42 (m, 2H, H-C4), 2.45-2.57 and 2.62-2.72 and 3.38-3.41 (m, 2H, H-C5), 2.96-3.18 (m, 2H, H-C3, H-C4a), 3.42-3.92 (m,2H, H-C2), 3.80 (s,3H, OCH₃), 4.09-4.30 (m, 2H, H-C10), 6.79-6.86 (m, 1H, H-C7), 6.87-6.95 (m, 1H, H-C9), 7.20-7.28 (m, 1H, H-C8).

d) rac-(3R,4aR,10aR) and rac-(3S,4aR,10aR)-6-Methoxy-1,2,3,4,4a,5,10,10a-octahydro-benzo[.g.]quinoline-3-carboxylic acid methyl ester p-toluenesulfonic acid salt

6-Methoxy-2,3,4,4a,5,10-hexahydro-benzo[.g.]quinoline-3-carboxylic acid hydrochloride (29.6 g, 100 mmol) is dissolved in methanol (592 ml) and cooled to −70°. Then NaBH₄ (5.68 g, 150 mmol) is added portionwise, so that the temperature does not rise above −65°. After the addition is complete the mixture is stirred for an additional 2 h, then warmed to −30° and poured on a solution of sulfuric acid (32.3 g) in methanol (125 ml). The reaction mixture is heated to reflux for 3.5 h. Then the methanol is evaporated and from the residue an aqueous work up is done (ethylacetate/water/Na₂CO₃; pH>9). The ethylacetate is evaporated, the residue dissolved again in ethylacetate and the two diastereomers are precipitated at 70° as their p-toluenesulfonic acid salts, by adding a solution of p-toluenesulfonic acid (17.1 g, 90 mmol) in ethylacetate (150 ml). The suspension is seeded with the product mixture, cooled down in the ice bath, filtered and washed with cold ethylacetate. The product is dried in vacuo at 60°. 35.1 g (78.4%). HPLC: about 1:1 diastereomeric mixture (99.4% area), assay (titration: 99.0%). ¹H-NMR (CDCl₃: 400 Mhz): rac-(3R,4aR,10aR) isomer: free base: 1.38-1.62 (m,2H, 4ax and 4a), 1.88 (br. s, 1H, NH), 2.15-2.33 (m, 2H, 4 eq, 5ax), 2.57-2.62 (in, 3H, (3ax,10ax,10a), 2.81-2.90 (m, 1H, 2ax), 2.92-3.05 (m, 2H, 5eq, 10 eq), 3.37-3.46 (m, 1H, 2 eq), 3.72 (s, 3H, COOCH₃), 3.84 (s, 3H, OCH₃), 6.67-6.78 (m, 2H,H7,H9), 7.09-7.15 (m,1H, H₈). rac-(3S,4aR,10aR) isomer: free base: 1.48-1.69 (m, 2H, 4ax, 4a), 2.03-2.20 (m, 2H, NH, 5ax), 2.38-2.47 (m, 1H, 4 eq), 2.57-2.74 (m, 3H, 3eq, 10ax, 10a), 2.90-3.05 (m, 3H, 2ax, 5eq, 10 eq), 3.54-3.61 (m, 1H, 2eq), 3.76 (s, 3H, COOCH₃), 3.84 (s, 3H, OCH₃), 6.67-6.76 (m, 2H, H7,H9), 7.08-7.15 (m, 1H, H8).

e) (3R,4aR,10aR) 6-Methoxy-1-methyl-1,2,3,4,4a,5, 10,10a-octahydro-benzo[.g.]quinoline-3-carboxylic acid methyl ester camphorsulfonic acid salt

A 1:1 mixture of rac-(3R,4aR,10aR) and rac-(3S,4aR,10aR)-6-Methoxy-1,2,3,4,4a,5,10,10a-octahydro-benzo[.g.]quinoline-3-carboxylic acid methyl ester p-toluenesulfonicacid salt (22.4 g, 50 mmol), acetic acid (10 ml), 37% formaldehyde (aq., 5.0 g, 62 mmol), 2.5 g Pd/C (10%) in methanol (225 ml) is hydrogenated at normal pressure at 60° until no more hydrogen is consumed. The catalyst is filtered off and the filtrate together with the washing is evaporated to a volume of 250 ml. To this solution 5.4 M sodium methylate solution in methanol (65 ml, 350 mmol) is added and the mixture is heated to reflux until the ester is completely hydrolysed (3 h). Then 68.6 g sulfuric acid are added and stirring at reflux is continued for another 6 h. To this reesterified product mixture is again added 5.4 M sodium methylate solution in methanol (250 ml, 1350 mmol) and after complete hydrolysis (3 h), sulfuric acid (69 g), whereby the mixture is heated at reflux for an additional 6 h. The methanol is evaporated at reduced pressure and from the residue an aqueous work up is done (ethylacetate/water/NaOH/Na₂CO₃ pH >9). The ethylacetate phase is evaporated completely. An HPLC analysis shows a ratio of 84:7 mixture in favour of the desired racemic (3R, 4aR,10aR) compound. 12.8 g (88%). This residue is dissolved at 65° in a mixture of isopropanol (42 ml) and ethylacetate (21 ml). To this hot solution a solution of (+)-camphorsulfonic acid (5.23 g, 22.5 mmol) in isopropanol (21 ml) is added. The mixture is slowly cooled down (3 h) to room temperature and finally to 0°. The precipitated salt is filtered off, washed with a mixture of cold isopropanol/ethylacetate and dried in vacuo at 55°. 8.0 g (31% from rac-(3R,4aR,10aR) and rac-(3S,4aR,10aR)-6-Methoxy-1,2,3,4,4a,5,10,10a-octahydro-benzo[.g.]quinoline-3-carboxylic acid methyl ester p-toluenesulfonicacid salt). HPLC purity 97.8%, enantiomeric ratio: 93:6.5 (HPLC).

f) (3R,4aR,10aR) 6-Methoxy-1-methyl-1,2,3,4,4a,5,10,10a-octahydro-benzo[g]quinoline-3-carboxylic acid

The free base is liberated from 6 g (11.5 mmol) of (3R,4aR,10aR) 6-Methoxy-1-methyl-1,2,3,4,4a,5,10,10a-octahydro-benzo[g]quinoline3-carboxylic acid methyl ester camphorsulfonic acid salt (toluene/water/Na₂CO₃ pH >9) and the toluene phase is evaporated to dryness. To the residue isopropanol (10 ml), water (40 g) and NaOH (0.48 g, 12 mmol) are added and the mixture is heated to reflux for 3 h. Then the pH is adjusted to pH 5 by adding 15% sulfuric acid. The product precipitates, is filtered and washed with water after the suspension was cooled to 5°. The product is dried at 80° in vacuo. 2.9 g (92%). ¹H-NMR (CD₃OD/NaOD: 400 Mhz): 1.13-1.27 (m, 1H, 4ax), 1.25-1.38 (m, 1H, 4a), 1.75-1.85 (m, 1H, 10 a), 1.96-2.25 (ml, 3H, 2ax, 4eq, 5ax), 2.30 (s, 3H, NCH₃), 2.42-2.53 (m, 2H, 3ax, 10ax), 2.82-2.95 (m, 1H, 5eq), 3.00-3.12 (m, 2H, 2eq, 10 eq), 3.68 (s, 3H, OCH₃), 6.57-6.62 (m, 2H, H7,H9), 6.92-7.00 (m, 1H, H8).

g) [3R,4aR,10aR]-3-(endo-3-Benzhydryloxy-8-aza-bicyclo[3.2.1]oct-8-yl)-6-methoxy-1-methyl-1,2,3,4,4a,5, 10,10a-octahydro-benzo[g]quinolin-3-yl)-methanone

[3R, 4aR, 10aR]-6-methoxy-1-methyl-1,2,3,4,4a,5,10,10a-octahydro-benzo[g]quino-line-3-carboxylic acid (3.7 g, 13.45 mmol) or sodium [3R,4aR,10aR]-6-methoxy-1-methyl-1,2,3,4,4a,5,10,10a-octahydro-benzo[g]quinoline3-carboxylate (4 g, 13.45 mmol, prepared from the above methyl ester with 1M NaOH in MeOH/THF 1:1 at room temperature and precipitated with MTBE) is suspended in dry DMF (150 ml) and THF (50 ml) at 0°. N-(3-Dimethylaminopropyl)-N′-ethylcarbodiimid hydrochloride (2.84 g, 14.8 mmol) and hydroxybenzotriazole (2 g, 14.8 mmol) are added and the solution is stirred for 90 min. Endo-3-benzhydryloxy-8-aza-bicyclo[3.2.1]octane (3.95 g, 13.45 mmol) in THF (50 ml) is added and the solution is kept at room temperature for 24 hours. The reaction mixture is quenched with sat. NaHCO₃, diluted with toluene/ethyl acetate 1:1 and washed carefully with water and brine. The organic phase is dried over sodium sulfate, filtered and concentrated in vacuo. White solid 6.1 g (11 mmol, 82%). M.P. 248-250° (free base). TLC 0.27 (silica, 8:1:1 cyclohexanes:toluene:EtOH/NH₄OH(95:5)), ESI-MS: 550. [α]_{D}−86.9 (c=1.02, dichloromethane).

h) [3S,4aR,10aR]-3-(endo-3-Benzhydryloxy-8-aza-bicyclo[3.2.1]oct-8-ylmethyl)-6-methoxy-1-methyl-1,2,3,4,4a,5,10,10a-octahydro-benzo[g]quinoline

To a solution of the above amide (6.07 g, 11.02 mmol) in THF (150 ml) is added lithium aluminum hydride (1.25 g, 33.06 mmol) at room temperature. After 1 day sat. potassium carbonate solution (6.2 ml) is added followed by 2 spatula of hyflo. After 1 hour the white suspension is filtered and washed with THF. The filtrate is diluted with ethyl acetate (300 ml) and washed with water and brine, the organic phase is dried over sodium sulfate, filtered and concentrated in vacuo. The yellowish foam is recrystallized once from ethanol. 4.97 g (9.25 mmol, 84%). M.p. 118-122° (free base). TLC 0.46 (silica, 8:1:1 cyclohexanes:toluene:EtOH/NH₄OH (95:5)). ESI-MS: 537.4. [MH]⁺. [α]D −70.3 (c=1.08, methanol).

### EXAMPLE 7

### [3R,4aS,10aS]-3-(endo-3-Benzhydryloxy-8-aza-bicyclo[3.2.1]oct-8-ylmethyl)-6-methoxy-1-methyl-1,2,3,4,4a,5,10,10a-octahydro-benzo[g]quinoline

Prepared analogously to Example 6, using sodium [3S,4aS,10aS]-&methoxy-1-methyl-1,2,3,4,4a,5,10,10a-octahydro-benzo[g]quinoline-3-carboxylate. M.p. 118-122° (free base). ESI-MS: 537 [MH]⁺. [α]_{D}+70.0 (c=1.05, MeOH).

The following compounds of formula I are prepared analogously to Example 6:

### EXAMPLE 8

### [3S,4aR,10aR]-3-[4-(1-H-Indol-3-yl)-piperidin-1-ylmethyl]-6-methoxy-1-methyl-1,2,3,4,4a,5,10,10a-octahydro-benzo[g]quinoline

M.p. 220-223° (free base). ESI-MS: 444 [MH]⁺. [α]_{D}−85.1 (c=1.12, DMF).

### EXAMPLE 9

### [3S,4aR,10aR]-4-(4-Chloro-phenyl)-1-(6-methoxy-1-methyl-1,2,3,4,4a,5,10,10a-octahydro-benzo[g]quinolin-3-ylmethyl)-piperidin-4-ol

M.p. 202-204° (free base). ESI-MS: 455 [MH]⁺. [α]_{D}−86.8 (c=0.825, DMF).

### EXAMPLE 10

### [3S,4aR,10aR]-3-(4-Benzhydryl-piperazin-1-ylmethyl)-6-methoxy-1-methyl-1,2,3,4,4a,5,10,10a-octahydro-benzo[g]quinoline

M.p. 278-280° (naphthalene-1,5-disulphonate). ESI-MS: 496 [MH]⁺. [α]_{D}−37.7 (c=0.79, DMF).

### EXAMPLE 11

### [3S,4aR,10aR]-3-[4,4-Bis-(4-methoxy-phenyl)-piperidin-1-ylmethyl]-6-methoxy-1-methyl-1,2,3,4,4a,5,10,10a-octahydro-benzo[g]quinoline

M.p. 216-219° (fumarate). ESI-MS: 541 [MH⁺]. [α]_{D}−51.2 (c=0.755, DMF).

### EXAMPLE 12

### N-[1-((3S,4aR,10aR)-6-Methoxy-1-methyl-1,2,3,4,4a,5,10,10a-octahydro-benzo[g]quinolin-3-ylmethyl)-piperidin-4-yl]-2,2-diphenyl-acetamide

M.p. 219-222° (free base). EI-MS: 537 [M]⁺. [α]_{D}−79.1 (c=1.09, DMF).

### EXAMPLE 13

### [1-((3S,4aR,10aR)-6-Methoxy-1 -methyl-1,2,3,4,4a,5,10,10a-octahydro-benzo[g]quinolin-3-ylmethyl)-piperidin-4-yl]-diphenyl-methanol

M.p. 100 118° (free base). CI-MS: 511 [MH]⁺. [α]_{D}−68.3 (c=1.02, DMF).

### EXAMPLE 14

### (3S,4aR,10aR)-3-{endo-3-[Bis-(4-fluoro-phenyl)-methoxy]-8-aza-bicyclo[3.2.1]oct-8-ylmethyl}-6-methoxy-1-methyl-1,2,3,4,4a,5,10,10a-octahydro-benzo[g]quinoline

M.p. 240-248° (naphthalene-1,5-disulfonate). ESI-MS: 573 [MH]⁺. [α]_{D}−34.8 (c=0.996, DMF).

### EXAMPLE 15

### [6aR,9R]-9-[2-(endo-3-Benzhydryloxy-8-aza-bicyclo[3.2.1]oct-8-yl)-ethyl]-7-methyl-4,6,6a,7,8,9-hexahydro-indolo[4,3-fg]quinoline

a) To a suspension of homolysergic acid (7.6 g, 27 mmol) in DMF (200 ml) is added pyridine (48 ml) and propanephosphonic acid anhydride (50% in DMF, 48 ml) at room temperature. After 10 hours endo-3-benzhydryloxy-8-aza-bicyclo[3.2.1]octane (7.9 g, 27 mmol) in THF (20 ml) is added. After 3 days toluene (500 ml) is added and the reaction mixture is concentrated in vacuo to about 150 ml. A second portion of toluene (500 ml) is added and concentrated again to about 150 ml. The resulting solution is poured onto iced water (500 ml) and made alkaline with ammonia. The resulting grey precipitate is filtered, washed with water and dried in the oven. The crude product is recrystallized from chloroform:MeOH 1:1. 9.76 g (65%). M.p. 246-252°. ESI-MS: 558 [MH]⁺. [α]_{D}+59.5 (c=0.985, chloroform:MeOH 1:1).

b) The above amide (9.76 g, 17.5 mmol) is added portionwise to a suspension of lithium aluminum hydride (2 g, 52 mmol) in THF (235 ml) at room temperature under argon atmosphere. After 20 hours at room temperature sat. potassium carbonate solution (10.5 ml) is added carefully under cooling. After 2 hours hyflo is added and the reaction mixture is filtered, washed with THF and the filtrate is concentrated in vacuo. The crude oil is dissolved in hot ethyl acetate/tert.-butyl-methylether, decolorized with activated charcoal and filtered. The filtrate is reduced in volume until the first crystals appear and put aside for crystallization. 6.7 g (70%). M.p. 165-166° . ESI-MS: 544 [MH]⁺. [α]_{D}+36.5 (c=1.14, MeOH).

The following compounds of formula I are prepared analogously to Example 15:

### EXAMPLE 16

### [6aR,9R]-9-{2-[4,4-Bis-(4-methoxy-phenyl)-piperidin-1-yl]-ethyl}-7-methyl-4,6,6a,7,8,9-hexahydro-indolo[4,3-fg]quinoline

M.p. 184-187° (free base, decomposition). ESI-MS: 548 [MH]⁺. [α]_{D}+40.2 (c=1.03, DMF).

### EXAMPLE 17

### [6aR,9R]-9-(2-[4-(1-H-Indol-3-yl)-piperidin-1-yl]-ethyl)-7-methyl-4,6,6a,7,8,9-hexahydro-indolo[4,3-fg]quinoline

M.p. 184-187° (EtOH, free base, decomposition). ESI-MS: 451 [MH]⁺. [α]_{D}+42.5 (c=1.07, chloroform).

### EXAMPLE 18

### [6aR,9R]-4-(4-Chloro-phenyl)-1-[2-(7-methyl-4,6,6a,7,8,9-hexahydro-indolo[4,3-fg)quinolin-9-yl)-ethyl]-piperidin-4-ol

M.p. 154-157° (ethyl acetate, free base). ESI-MS: 464, 462 [MH]⁺. [α]_{D}+38.3 (c=1.01, DMF).

### EXAMPLE 19

### [6aR,9R]-9-[2-(4-Benzhydryl-piperazin-1-yl)-ethyl]-7-methyl-4,6,6a,7,8,9-hexahydro-indolo[4,3-fg]quinoline

M.p. 165-169° (ethyl acetate, free base). ESI-MS: 503 [MH]⁺. [α]D+40.5 (c=1.01, MeOH).

### EXAMPLE 20

### [6aR,9S]-9-(endo-3-Benzhydryloxy-8-aza-bicyclo[3.2.1]oct-8-ylmethyl)-7-methyl-4,6,6a,7,8,9-hexahydro-indolo[4,3-fg]quinoline

A mixture of lysergol-8-methane sulphonate (4.19 g, 12.61 mmol) and endo-3-benzhydryloxy-8-aza-bicyclo[3.2.1]octane (7.4 g, 25.22 mmol) in dimethylacetamide (8.4 ml) is heated at 125° under argon for 1 hour. The dark reaction mixture is diluted with ethyl acetate (400 ml) and washed with 2 N NaOH, water and brine. The organic phase is dried over sodium sulfate, decolorized with activated charcoal, filtered and concentrated in vacuo. Flash chromatography (silica, ethyl acetate+1% ammonia, then ethyl acetate:EtOH:ammonia 9:1:0.1) yields a crude compound that is triturated with pentane, filtered and washed with pentane and finally dried at 120° in high vac. 3.07 g (5.8 mmol, 46%). M.p. 173° (dec.). ES]-MS: 530 [MH]⁺. [α]_{D}+17.5 (c=0.4, MeOH).

The following compound of formula I is prepared analogously to Example 20.

### EXAMPLE 21

### [6aR,9S]-9-(4-Benzhydryl-piperazin-1-ylmethyl)-7-methyl-4,6,6a,7,8,9-hexahydro-indolo[4,3-fg]quinoline

M.p. >200° (free base, decomposition). ESI-MS: 489 [MH]⁺. [α]_{D}+30.7 (c=0.815, MeOH).

The following compounds of formula I are prepared analogously to Example 20, using 1-methyl-lysergol-8-methane sulphonate.

### EXAMPLE 22

### [6aR,9S]-4-(4-Chloro-phenyl)-1-(4,7-dimethyl4,6,6a,7,8,9-hexahydro-indolo[4,3-fg]quinolin-9-ylmethyl)-piperidin-4-ol

M.p. 101-107° (tert.-butyl-methylether). ESI-MS: 462 [MH]⁺. [α]_{D}+35.4 (c=1.025, chloroform).

### EXAMPLE 23

### [6aR,9S]-9-(endo-3-Benzhydryloxy-8-aza-bicyclo[3.2.1]oct-8-ylmethyl)-4,7-dimethyl-4,6,6a,7,8,9-hexahydro-indolo[4,3-fg]quinoline

M.p. 172-175° (free base). ESI-MS: 544 [MH]⁺. [α]_{D}+20.8 (c=0.845, DMF).

### EXAMPLE 24

### [6aR,9S]-9-(4-Benzhydryl-piperazin-1-ylmethyl)-4,7-dimethyl-4,6,6a,7,8,9-hexahydro-indolo[4,3-fg]quinoline

M.p. 172-175° (free base). ESI-MS: 503 [MH]⁺. [α]_{D}+18.9 (c=1.04, DMF).

The following compounds of formula I are prepared analogously to Example 20, using 2-chlorolysergol-8-methane sulphonate, prepared as follows:

To a suspension of lysergol-8-methane sulfonate (5 g, 15 mmol) in acetonitrile (290 ml) is added boron trifluoride diethyl etherate (7.25 ml) at −5° under inert atmosphere (N₂) followed by the addition of sulfurylchloride (1.35 ml) in dichloromethane (115 ml) at the same temperature. After 1 hour the solution is quenched with 2M ammonia (100 ml), diluted with dichloromethane (200 nm) and washed with water and brine. The organic phase is dried with sodium sulfate and treated with activated charcoal, filtred and concentrated in vacuo. Silica gel chromatography (ethyl acetate:dichloromethane 1:1) of the concentrate affords 3.3 g (9 mmol, 60%) of the compound. m.p. 125° (broad, decomposition). EI-MS: 366.

### EXAMPLE 25

### [6aR,9S]-9-[4,4-Bis-(4-methoxy-phenyl)-piperidin-1-ylmethyl]-5-chloro-7-methyl-4,6,6a,7,8,9-hexahydro-indolo[4,3-fg]quinoline

M.p. >204° (free base, decomposition). ESI-MS: 570, 568 [MH]⁺. [α]_{D}+38.4 (c=1.01, dichloromethane).

### EXAMPLE 26

### [6aR,9S]-1-(5-Chloro-7-methyl-4,6,6a,7,8,9-hexahydro-indolo[4,3-fg]quinolin-9-ylmethyl)-4-(4-chloro-phenyl)-piperidin-4-ol

M.p. >192° (free base, decomposition). FAB-MS: 486, 484, 482 [MH]⁺. [α]_{D}+23.1 (c=0.935, DMF).

### EXAMPLE 27

### [6aR,9S]-9-(endo-3-Benzhydryloxy-8-aza-bicyclo[3.2.1]oct-8-ylmethyl)-5-chloro-7-methyl-4,6,6a,7,8,9-hexahydro-indolo[4,3-fg]quinoline

M.p. 164-169° (m-tartrate). FAB-MS: 564 [MH]⁺. [α]_{D}+20.5 (c=1.105, pyridine).

### EXAMPLE 28

### [6aR,9S]-9-(4-Benzhydryl-piperazin-1-ylmethyl)-5-chloro-7-methyl4,6,6a,7,8,9-hexahydro-indolo[4,3-fg]quinoline

M.p. 208° (free base, decomposition). FAB-MS: 525, 523 [MH]⁺. [α]_{D}+26.9 (c=1.01, dichloromethane).

The following compounds of formula I are prepared analogously to Example 20, using 9,10-dihydrolysergol-8-methane sulphonate.

### EXAMPLE 29

### [6aR,9S,10aR]-9-(endo-3-Benzhydryloxy-8-aza-bicyclo[3.2.1]oct-8-ylmethyl)-7-methyl-4,6,6a,7,8,9,10,10a-octahydro-indolo[4,3-fg]quinoline

M.p. 137-142° (ethyl acetate, free base). ESI-MS: 532 [MH]⁺. [α]_{D}−48.0 (c=1.03, MeOH).

### EXAMPLE 30

### [6aR,9S,10aR]-9-(4-Benzhydryl-piperazin-1-ylmethyl)-7-methyl-4,6,6a,7,8,9,10,10a-octahydro-indolo[4,3-fg]quinoline

M.p. 230-233° (ethyl acetate, free base). ESI-MS: 491 [MH]^{+. [α]}_{D}−45.5 (c=1.05, dichloromethane).

The following compounds of formula I are prepared analogously to Example 20, using 2chloro-9,10-dihydrolysergol-8-methane sulphonate:

### EXAMPLE 31

### [6aR,9S,10aR]-9-(endo-3-Benzhydryloxy-8-aza-bicyclo[3.2.1]oct-8-ylmethyl)-5-chloro-7-methyl-4,6,6a,7,8,9,10,10a-octahydro-indolo[4,3-fg]quinoline

M.p. 162-168° (EtOH, 1.4xfumarate). ESI-MS: 566 [MH]⁺. [α]_{D}−42.8 (c=0.85, DMF).

### EXAMPLE 32

### [6aR,9S,10aR]-9-(4-Benzhydryl-piperazin-1-ylmethyl)-5-chloro-7-methyl-4,6,6a,7,8,9,10,10a-octahydro-indolo[4,3-fg]quinoline

M.p. 250° (ethyl acetate, free base, decomposition). ESI-MS: 525 [MH]⁺. [α]_{D}−57.7 (c=0.96, chloroform).

The following compounds of formula I are prepared analogously to Example 20, using 2-bromo-9,10-dihydrolysergol-8-methane sulphonate, prepared as follows:

To a suspension of 9,10-dihydrolysergol-8-methane sulfonate (10 g, 29.9 mmol) in dry THF (400 ml) is added tris-2-pyrrolidone-perbromide hydrobromide (20 g, 40 mmol) dissolved in THF (100 ml) at room temperature. After 24 hours the reaction mixture is made alkaline with 2N ammonia and is diluted with ethyl acetate (300 ml). The organic phase is washed with water and brine, dried over sodium sulfate, treated with activated charcoal, filtered and concentrated in vacuo. The crude product is recrystallized from ethyl acetate. 8.2 g (19.8 mmol, 66%). m.p. 169-171°. TLC 0.4 (silica, toluene:EtOH 5:1).

### EXAMPLE 33

### [6aR,9S,10aR]-9-(endo-3-Benzhydryloxy-8-aza-bicyclo[3.2.1]oct-8-ylmethyl)-5-bromo-7-methyl-4,6,6a,7,8,9,10,10a-octahydro-indolo[4,3-fg]quinoline

M.p. 109-114° (free base). ESI-MS: 612, 610 [MH]⁺. [α]_{D}−61.3 (c=0.945, DMF).

### EXAMPLE 34

### [6aR,9S,10aR]-9-(4-Benzhydryl-piperazin-1-ylmethyl)-5-bromo-7-methyl-4,6,6a,7,8,9,10,10a-octahydro-indolo[4,3-fg]quinoline

M.p. 255° (ethyl acetate, free base, decomposition). ESI-MS: 571, 569 [MH]⁺. [α]_{D}−57.3 (c=1.005, chloroform).

The following compounds of formula I are prepared analogously to Example 20, using 6-ethyl-9,10-dihydrolysergol-8-methane sulphonate.

### EXAMPLE 35

### [6aR,9S,10aR]-9-(endo-3-Benzhydryloxy-8-aza-bicyclo[3.2.1]oct-8-ylmethyl)-7-ethyl-4,6,6a,7,8,9,10,10a-octahydro-indolo[4,3-fg]quinoline

M.p. 175-178° (free base). ESI-MS: 546 [MH]⁺. [α]_{D}−42.7 (c=0.985, chloroform).

### EXAMPLE 36

### [6aR,9S,10aR]-9-(4-Benzhydryl-piperazin-1-ylmethyl)-7-ethyl-4,6,6a,7,8,9,10,10a-octahydro-indolo[4,3-fg]quinoline

M.p. 203-205° (isopropanol, free base). ESI-MS: 505 [MH]⁺. [α]_{D}−44.6 (c=0.985, chloroform).

The following compounds of formula I are prepared analogously to Example 20, using homo-9,10-dihydrolysergol-8-methane sulphonate.

### EXAMPLE 37

### [6aR,9S,10aR]-9-[2-(endo-3-Benzhydryloxy-8-aza-bicyclo[3.2.1]oct-8-yl)-ethyl]-7-methyl-4,6,6a,7,8,9,10,10a-octahydro-indolo[4,3-fg]quinoline

M.p. 168-169° (tert.-butyl-methylether, free base). ESI-MS: 546 [MH]⁺. [α]_{D}−44.2 (c=1.06, chloroform).

### EXAMPLE 38

### [6aR,9S,10aR]-9-[2-(4-Benzhydryl-piperazin-1-yl)-ethyl]-7-methyl-4,6,6a,7,8,9,10,10a-octahydro-indolo[4,3-fg]quinoline

M.p. 206-212° (free base). ESI-MS: 505 [MH]⁺. [α]_{D}−51.5 (c=1.13, MeOH).

The following compounds of formula I are prepared analogously to Example 20, using 10α-methoxy-lumilysergol-8-methane sulphonate.

### EXAMPLE 39

### [6aR,9S,10aS]-9-[4,4-Bis-(4-methoxy-phenyl)-piperidin-1-ylmethyl]-10a-methoxy-7-methyl-4,6,6a,7,8,9,10,10a-octahydro-indolo[4,3-fg]quinoline

M.p. 215-222° (ethyl acetate/MeOH, free base). ESI-MS: 566 [MH]⁺. [α]_{D}−1.8 (c=1.03, DMF).

### EXAMPLE 40

### [6aR,9S,10aS]-9-[4-(1-H-Indol-3-yl)-piperidin-1-ylmethyl]-10a-methoxy-7-methyl-4,6,6a,7,8,9,10,10a-octahydro-indolo[4,3-fg]quinoline

M.p. 222-227° (isopropanol, free base). ESI-MS: 469 [MH]⁺. [α]D−2.3 (c=1.03, DMF).

### EXAMPLE 41

### [6aR,9S,10aS]-4-(4-Chloro-phenyl)-1-(10a-methoxy-7-methyl-4,6,6a,7,8,9,10,10a-octahydro-indolo[4,3-fg]quinolin-9-ylmethyl)-piperidin-4-ol

M.p.144-148° (ethyl acetate, free base). ESI-MS: 482, 480 [MH]⁺. [α]D−8.7 (c=1.04, DMF).

### EXAMPLE 42

### [6aR,9S,10aS]-1-(10a-Methoxy-7-methyl-4,6,6a,7,8,9,10,10a-octahydro-indolo[4,3-fg]quinolin-9-ylmethyl)-4-(3-trifluoromethyl-phenyl)-piperidin-4-ol

M.p.135-140° (ethyl acetate/cyclohexane, free base). ESI-MS: 514 [MH]⁺. [α]_{D}−2.3 (c=O.97, chloroform).

### EXAMPLE 43

### [6aR,9S,10aS]-4-(4-Chloro-3-trifluoromethyl-phenyl)-1-(10a-methoxy-7-methyl-4,6,6a,7,8,9,10,10a-octahydro-indolo[4,3-fg]quinolin-9-ylmethyl)-piperidin-4-ol

M.p.143-146° (ethyl acetate/cyclohexane, free base). ESI-MS: 550, 548 [MH]⁺. [α]_{D}−5.9 (c=1.0, chloroform).

### EXAMPLE 44

### [6aR,9S,10aS]-9-(endo-3-Benzhydryloxy-8-aza-bicyclo[3.2.1]oct-8-ylmethyl)-10a-methoxy-7-methyl4,6,6a,7,8,9,10,10a-octahydro-indolo[4,3-fg]quinoline

M.p.238-243° (dichloromethane/MeOH, free base). ESI-MS: 562 [MH]⁺. [α]_{D}−4.9 (c=0.975, DMF).

### EXAMPLE 45

### [6aR,9S,10aS]-9-(4-Benzhydryl-piperazin-1-ylmethyl)-10a-methoxy-7-methyl- 4,6,6a,7,8,9,10,10a-octahydro-indolo[4,3-fg]quinoline

M.p.:glassy residue ESI-MS: 521 [MH]⁺. [α]_{D}−9.2 (c=1.075, chloroform).

The following compounds of formula I are prepared analogously to Example 20, using homo-10α-methoxy-lumilysergol-8-methane sulphonate prepared as follows:

To [6aR,9R,10aS]-2-(10a-methoxy-7-methyl-4,6,6a,7,8,9,10,10a-octahydro-indolo[4,3-fg]quinolin-9-yl)ethanol (6.2 g, 20.6 mmol in dry pyridine (100 ml) is added methanesulfonyl chloride (4.8 ml, 62 mmol) at 0°. After 2.5 hours at room temperature the reaction mixture is made alkaline with sat. K₂CO₃ solution. The resulting solution is diluted with ethyl acetate, washed with water and brine, dried over sodium sulfate, filtered and concentrated in vacuo. Residual pyridine is removed with repeated treatment with toluene on the rotary evaporator. The brownish powder is triturated with diisopropylether, filtered and dried at 70° at reduced pressure. 6.04 g (15.98 mmol, 77%). mp. 124-128° (broad). ESI-MS: 379.

### EXAMPLE 46

### [6aR,9R,10aS]-9-{2-[4,4-Bis-(4-methoxy-phenyl)-piperidin-1-yl]-ethyl}-10a-methoxy-7-methyl-4,6,6a,7,8,9,10,10a-octahydro-indolo[4,3-fg]quinoline

M.p. 129-136° (ethyl acetate/pentane, free base). ESI-MS: 580 [MH]⁺. [α]_{D}−12.0 (c=1.04, MeOH).

### EXAMPLE 47

### [6aR,9R,10aS]-9-{2-[4-(1-H-Indol-3-yl)-piperidin-1-yl]-ethyl}-10a-methoxy-7-methyl-4,6,6a,7,8,9,10,10a-octahydro-indolo[4,3-fg]quinoline

M.p. 153-156° (ethyl acetate/pentane, free base). ESI-MS: 483 [MH]⁺. [α]_{D}−12.2 (c=0.995, MeOH).

### EXAMPLE 48

### [6aR,9R,10aS]-4-(4-Chloro-phenyl)-1-[2-(10a-methoxy-7-methyl-4,6,6a,7,8,9,10,10a-octahydro-indolo[4,3-fg]quinolin-9-yl)-ethyl]-piperidin-4-ol

M.p. 133-139° (ethyl acetate/pentane, free base). ESI-MS: 496, 494 [MH]⁺. [α]_{D}−12.6 (c=1.03, MeOH).

### EXAMPLE 49

### [6aR,9R,19aS]-9-[2-(endo-3-Benzhydryloxy-8-aza-bicyclo[3.2.1]oct-8-yl)-ethyl]-10a-methoxy-7-methyl-4,6,6a,7,8,9,10,10a-octahydro-indolo[4,3-fg]quinone

M.p. 156-165° (EtOH, 1.5 fumarate). ESI-MS: 576 [MH]⁺. [α]_{D}+2.4 (c=0.84, DMF).

### EXAMPLE 50

### [6aR,9R,10aS]-9-[2-(4-Benzhydryl-piperazin-1-yl)-ethyl]-10a-methoxy-7-methyl-4,6,6a,7,8,9,10,10a-octahydro-indolo[4,3-fg]quinoline

M.p. 130° (ethyl acetate/pentane, broad, free base). ESI-MS: 535 [MH]⁺. [α]_{D}−10.4 (c=0.99, MeOH).

The following compounds of formula I are prepared analogously to Example 20, using 2-bromo 10α-methoxy-lumilysergol-8-methane sulphonate, prepared as follows:

To a solution of [6aR,9R,10aS]-methanesulfonic acid 10a-methoxy-7-methyl-4,6,6a,7,8,9,10,10a-octahydro-indolo[4,3-fg]quinolin-9-ylmethyl ester (1.82 g, 5 mmol) in dioxane (27 ml) is added N-bromosuccinimid (979 mg, 5.5 mmol) in small portions at room temperature. After 2.5 hours the reaction mixture is diluted with ethyl acetate and iced water, made alkaline with 2 M ammonia, washed with water and brine. The organic phase is dried over sodium sulfate, filtered and concentrated in vacuo. The crude product is filtered through basic aluminum oxide and eluted with ethyl acetate. Brown solid 1.98 g (4.47 mmol, 74%). m.p. 198° (decomposition).

### EXAMPLE 51

### [6aR,9S,10aS]-9-[4,4-Bis-(4-methoxy-phenyl)-piperidin-1-ylmethyl]-5-bromo-10a-methoxy-7-methyl-4,6,6a,7,8,9,10,10a-octahydro-indolo[4,3-fg]quinoline

M.p. 167-172° (EtOH, 1.5xtartrate). ESI-MS: 646, 644 [MH⁺]. [α]_{D}+17.8 (c=1.02, DMF).

### EXAMPLE 52

### [6aR,9S,10aS]-5-Bromo-9-[4-(1-H-indol-3-yl)-piperidin-1-ylmethyl]-10a-methoxy-7-methyl-4,6,6a,7,8,9,10,10a-octahydro-indolo[4,3-fg]quinoline

M.p. 160-170° (EtOH, di-tartrate, decomposition). ESI-MS: 549, 547 [MH]⁺. [α]_{D}+15.4 (c=0.995, DMF).

### EXAMPLE 53

### [6aR,9S,10a]-1-(5-Bromo-10a-methoxy-7-methyl-4,6,6a,7,8,9,10,10a-octahydro-indolo[4,3-fg]quinolin-9-ylmethyl)-4-(4-chloro-phenyl)-piperidin-4-ol

M.p. 150-160° (EtOH, di-tartrate, decomposition). ESI-MS: 560, 558 [MH]⁺. [α]_{D}+13.0 (c=1.035, DMF).

### EXAMPLE 54

### [6aR,9S,10aS]-9-(endo-3-Benzhydryloxy-8-aza-bicyclo[3.2.1]oct-8-ylmethyl)-5-bromo-10a-methoxy-7-methyl-4,6,6a,7,8,9,10,10a-octahydro-indolo[4,3-fg]quinoline

M.p. 204-208° (EtOH, di-tartrate, decomposition). ESI-MS: 642, 640 [MH]⁺. [α]_{D}+15.2 (c=0.995, DMF).

The following compound of formula I is prepared analogously to Example 20, using 2-phenyl-9,10-dihydrolysergol-8-methane sulphonate prepared as follows:

A mixture of 2-bromo-9,10-dihydrolysergol (0.6 g, 1.79 mmol), phenylboronic acid (0.25 g, 2.05 mmol), Pd(II)acetate (13 mg) and tri(o-tolyl)phosphin (28 mg) in toluene (50 ml), EtOH (0.9 ml) and 2M Na₂CO₃ (3 ml) is stirred at 90° under argon for 5 hours. The reaction mixture is diluted with ethyl acetate (250 ml) and washed with water and brine. The organic phase is dried over sodium sulfate, decolorized with activated charcoal, filtered and concentrated in vacuo. The yellow residue is recrystallized from methanol/tert.-butyl-methylether. White crystals 230 mg (0.7 mmol, 38%). mp. 204-211°.

To the above 2-phenyl-9,10-dihydrolysergol (230 mg, 0.69 mmol) in pyridine (10 ml) is added methanesulfonyl chloride (161 μl, 2.07 mmol) at 0°. After 1 hour at room temperature the greenish reaction mixture is made alkaline with 2M ammonia. The resulting solution is diluted with ethyl acetate, washed with water and brine, dried over sodium sulfate, filtered and concentrated in vacuo. Residual pyridine is removed with repeated treatment with toluene on the rotary evaporator. Yellowish oil 270 mg (0.66 mmol, 95%). FAB-MS: 411 (M+H)⁺.

### EXAMPLE 55

### [6aR,9S,10aR]-9-(endo-3-Benzhydryloxy-8-aza-bicyclo[3.2.1]oct-8-ylmethyl)-7-methyl-5-phenyl-4,6,6a,7,8,9,10,10a-octahydro-indolo[4,3-fg]quinoline

M.p. 115-135° (EtOH, free base). ESI-MS: 608 [MH]⁺. [α]_{D}−58.6 (c=0.915, DMF).

1. A compound of formula I 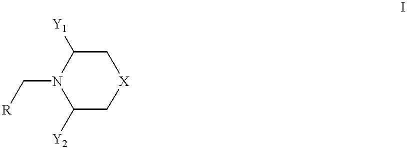
wherein
X is 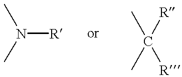
wherein R′ is a group (a) 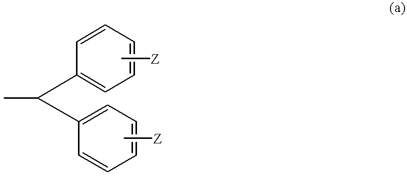
and either R″ is H or OH and R′″ is a group (b), (c) or (d) 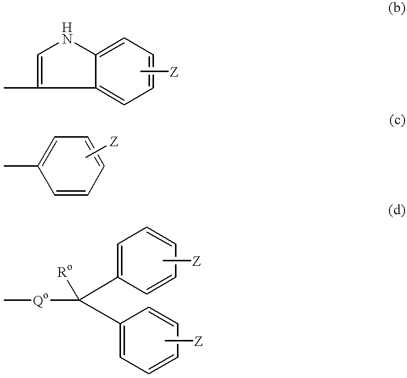
or R″ and R′″ each are a group (c),
wherein Z is H, halogen, trifluoromethyl, (C₁₋₄)alkyl or (C₁₋₄)alkoxy, Q° is —O—, —NH—CO— or a single bond and R° is hydrogen or hydroxy,
Y₁ and Y₂ are H or, when X is 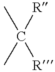
wherein R″ is H and R′″ is a group (d), Y₁ and Y₂ can also form together a —CH₂—CH₂— bridge, and
R is a group (e) or (f) 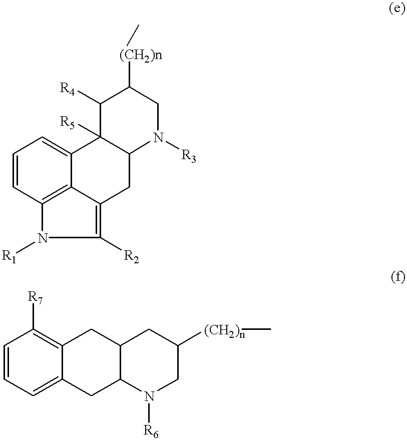
wherein
n is 0 to 3
R₁ is H, (C₁₋₄)alkyl or —SO₂—CH₃
R₂ is H, halogen, (C₁₋₄)alkyl, (C₁₋₄)alkoxy, (C₁₋₄)alkylthio or phenyl,
R₃ is H, (C₁₋₄)alkyl or a group (g) 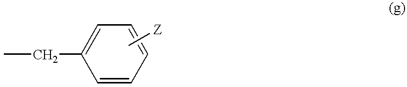
wherein Z is as defined above,
R₄ and R₅ each are H or together form a bond, or R₄ is H and R₅ is (C₁₋₄)alkoxy,
R₆ is (C₁₋₄) alkyl or a group (g) and
R₇ is (C₁₋₄) alkoxy,
provided that when X is 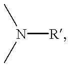
R is of formula (f) and n is 1, then Z is different from fluor in free base or acid addition salt form.

2. [3S,4aR,10aR]-3-(endo-3-Benzhydryloxy-8-aza-bicyclo[3.2.1]oct-8-ylmethyl)-6-methoxy-1-methyl-1,2,3,4,4a,5,10,10a-octahydro-benzo[g]quinoline in free base or acid addition salt form.

3. [3R,4aR,10aR]-3-(endo-3-Benzhydryloxy-8-aza-bicyclo[3.2.1]oct-8-ylmethyl)-6-methoxy-1-methyl-1,2,3,4,4a,5,10,10a-octahydro-benzo[g]quinoline in free base or acid addition salt form.

4. [6aR,9R]-9-[2-(endo-3-Benzhydryloxy-8-aza-bicyclo[3.2.1]oct-8-yl)-ethyl]-7-methyl-4,6,6a,7,8,9-hexahydro-indolo[4,3-fg]quinoline in free base or acid addition salt form.

5. A process for the preparation of a compound of formula I as defined in claim 1, or a salt thereof, which includes the step of
a) reducing a compound of formula II 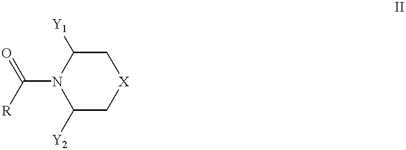
wherein X, Y₁, Y₂ and R are as defined in claim 1, or
b) reacting a compound of formula III 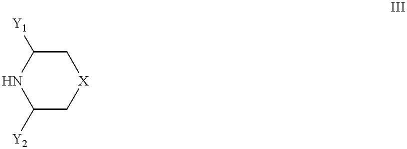
wherein X, Y₁ and Y₂ are as defined in claim 1, with a compound of formula IV
R—CH₂—Q IV
wherein R is as defined in claim 1 and Q is halogen, mesyl or tosyl,
and recovering the thus obtained compound of formula I in free base or acid addition salt form.

6. A compound of any one of claims 1 to 4 in free base or pharmaceutically acceptable acid addition salt form, for use as a pharmaceutical.

7. A compound of any one of claims 1 to 4 in free base or pharmaceutically acceptable acid addition salt form, for use in the treatment of any state resulting from Aβ accumulation or deposition in brain tissue.

8. A pharmaceutical composition comprising a compound of any one of claims 1 to 4 in free base or pharmaceutically acceptable acid addition salt form, in association with a pharmaceutical carrier or diluent.

9. The use of a compound of any one of claims 1 to 4 in free base or pharmaceutically acceptable acid addition salt form, as a pharmaceutical for the treatment of any state resulting from Aβ accumulation or deposition in brain tissue.

10. The use of a compound of any one of claims 1 to 4 in free base or pharmaceutically acceptable acid addition salt form, for the manufacture of a medicament for the treatment of any state resulting from Aβ accumulation or deposition in brain tissue.

11. A method for the treatment of any state resulting from Aβ accumulation or deposition in brain tissue, in a subject in need of such treatment, which comprises administering to such subject a therapeutically effective amount of a compound of any one of claims 1 to 4 in free base or pharmaceutically acceptable acid addition salt form
